(19) United States
(12) Patent Application Publication
(10) Pub. No.: US 2009/0318838 A1
(43) Pub. Date: Dec. 24, 2009

(54) Neoplastic cell destruction device and method utilizing low frequency sound waves to disrupt or displace cellular materials

(76) Inventor: Daniele Kenny, Miller Place, NY (US)

Correspondence Address: Bernard S. Hoffman, 10 Colgate Street, Port Jefferson Station, NY, 11776, US

(21) Appl. No.: 12/583,664

(22) Filed: Aug. 24, 2009

Related U.S. Application Data
(63) Continuation-in-part of application No. 11/134,011, filed on May 20, 2005.
Application No. 11/134,011 is a continuation of application No. 08/777,452, filed on Dec. 30, 1996, now Pat. No. 7,416,535.

(51) Int. Cl. A61N 7/00; A61B 8/00

(52) U.S. Cl. 601/2; 600/439

(57) ABSTRACT

A neoplasm cell destruction device utilizing low frequency sound waves to disrupt or displace cellular materials in neoplastic cells so as to damage and ultimately destruct the neoplastic cells. The device includes a plurality of signal generators, a controller, a plurality of amplifiers, a plurality of transducers, and a target interface. The controller is in electrical communication with, and generates timing and control signals for simultaneously activating, the plurality of signal generators. Each amplifier is in electrical communication with a respective signal generator and amplifies the signal generated by the respective signal generator so as to form an amplified signal. Each transducer is in electrical communication with a respective amplifier and is driven by the amplified signal formed by the respective amplifier. The target interface combines the waveforms formed by the plurality of transducers to form an interference wave that is a low frequency sound wave.

### 1. CROSS REFERENCE TO RELATED APPLICATIONS

The instant patent application is a Continuation-In-Part patent application of patent application Ser. No. 11/134,011, filed May 20, 2005, entitled NEOPLASM CELL DESTRUCTION DEVICE, incorporated herein by reference thereto, and which is a Continuation patent application of patent application Ser. No. 08/777,452, filed Dec. 30, 1996, entitled NEOPLASTIC CELL DESTRUCTION DEVICE AND METHOD UTILIZING LOW FREQUENCY SOUND WAVES TO DISRUPT OR DISPLACE CELLULAR MATERIALS, now U.S. Pat. No. 7,416,535 B1, issued on Aug. 26, 2008, and incorporated herein by reference thereto.

### 2. BACKGROUND OF THE INVENTION

A. Field of the Invention.

The embodiments of the present invention relate to a biological cell destruction device, and more particularly, the embodiments of the present invention relate to a neoplasm cell destruction device utilizing low frequency sound waves to disrupt or displace cellular materials in neoplastic cells.

B. Description of the Prior Art.

High frequency acoustic waves or ultrasound may be used to remotely heat industrial or biological materials. There has been strong evidence in research and clinical laboratories that focused ultrasound for cancer hyperthermia will become a useful mode of treating cancer patients in addition to the surgical, radiological, and chemotherapeutic methods that are available now.

In the treatment of tumors in cancer hyperthermia, focused ultrasound heats the tumor to a temperature of approximately 43° C. while the adjacent healthy tissue is kept at a lower temperature, closer to normal body temperature of 37° C. The elevated temperature in the tumor disrupts the tumor growth and eventually kills it. This allows the cancer to potentially be treated without surgery, without ionizing radiation, or without chemotherapy.

Conventional focused ultrasound for heating is employed by using either a scanned ultrasound transducer or a phased array. The scanned transducer uses a lens, much like an optical magnifying glass focus sunlight, while the phased array uses electronic delays among the array elements to achieve focusing. A burst of sound is then emitted, which converges at the focus to provide localized high intensity acoustic energy. Some of the high energy acoustic energy is absorbed by the tissue at the focus and is dissipated as concentrated focal heat. The rest of the energy travels through the focus and is slowly dissipated into the surrounding tissues as distributed heat.

Biomedical hyperthermia applicators using a plurality of sound sources to heat larger distributed volumes have also been investigated. These investigations have relied upon linear thermal superposition of the plurality of sound sources to heat the target tissue. Nonlinear effects of sound propagation through animal tissue and materials have also been studied for a single sound source.

It is generally recognized that the use of microwave energy to produce moderate internal heating is an effective tool in the treatment of tissue, especially neoplastic tumors. The primary factor limiting this treatment in the past has been the difficulty of delivering the heat to a target region below the skin surface. Of course, it is possible to use an interstitial source, but this method has the drawback of being invasive. Because of this limitation, noninvasive treatment has largely been confined to treatment of surface tumors, since it is difficult to heat deep tumors without also heating the intervening tissue.

In order to get significant heating in tumors more than a few millimeters below the skin surface, the field from a single source at the skin surface will have to be high and therefore painful. One approach has used a moving source that is generally activated by switching discrete sub-arrays of sources. The moving source, however, results in an incoherent summation of energy at the tumor site. While tending to reduce the heating effects in the intervening tissue, this method has not eliminated the heating of the intervening tissue or reduced it to an acceptable level.

Additionally, to insure that the desired volume of tissue is potentially heated, an operator must not only know the characteristics in the area of interest, but also be able to determine which tissues are being heated. The ability to make this determination depends on the use of an interstitial probe or a radiometer. This method also does not allow for imaging of the area, except to use other modalities, such as CT, MRI, ultrasound, etc. These methods, while noninvasive, do not provide appropriate characteristics of the area and tissue to maximize the heating of the target tissue with microwaves.

Most cancer cells during metastasis are rapidly killed by mechanical trauma associated with shape-transitions that requires increases in cell surface area.¹ The hypothesis has been advanced that these increases in surface area occur in two phases. First, there is an apparent increase as a result of surface unfolding that is reversible and non-lethal. Second, there is a true increase during which cell surface membranes are stretched, with an increase in membrane tension. When tension exceeds a critical level, the surface membranes rupture and the irreversible change is lethal. ¹ L. Weiss, J. P. Harlos, and G. Elkin; Int. J. Cancer 44; 143-148 (1989).

Numerous innovations for wave-related devices have been provided in the prior art, which will be described below in chronological order to show advancement in the art, and which are incorporated herein by reference thereto. Even though these innovations may be suitable for the specific individual purposes to which they address, however, they differ from the embodiments of the present invention in that they do not teach a cancer cell destruction device utilizing low frequency sound waves to disrupt or displace cellular materials in cancerous cells.

(1) U.S. Pat. No. 3,880,152 to Nohmura.

U.S. Pat. No. 3,880,152 issued to Nohmura on Apr. 29, 1975 in U.S. class 601 and subclass 47 teaches a chair or a bed having speakers incorporated therein. The speakers are disposed against the inside surfaces of the seat and back of the chair and the top surface of the bed so that the openings of the speakers will be directed toward a human body resting therein.

(2) U.S. Pat. No. 4,055,170 to Nohmura.

U.S. Pat. No. 4,055,170 issued to Nohmura on Oct. 25, 1977 in U.S. class 601 and subclass 47 teaches a health promoting apparatus including a chair, bed, or the like having a loudspeaker incorporated therein. An opening formed in the chair is closed by a pretensioned flexible sheet. The sound waves from the loudspeaker cause the flexible sheet to vibrate, thereby transmitting vibrations to a chair occupant.

(3) U.S. Pat. No. 4,315,514 to Drewes et al.

U.S. Pat. No. 4,315,514 issued to Drewes et al. on Feb. 16, 1982 in U.S. class 600 and subclass 427 teaches an ultrasound apparatus and a method for destroying selected cells in a host without damage to non-selected cells, which includes selecting a transmission path from an energy source to the selected cells, determining one or more of the resonant frequencies of the selected cells, selecting as a destructive frequency one of the resonant frequencies at which the transmissibility of the selected cells is higher than the transmissibility of the non-selected cells in the transmission path, and transmitting energy from the source at the destructive frequency along the path with sufficient intensity to destroy the selected cells without destroying the non-selected cells.

(4) U.S. Pat. No. 4,343,301 to Indech.

U.S. Pat. No. 4,343,301 issued to Indech on Aug. 10, 1982 in U.S. class 601 and subclass 3 teaches a method of generating a high energy density at any point in the body noninvasively by two high frequency sonic beams creating a low frequency beating pattern at their intersection locus. One method provides for two transducers at different angular positions. Each transducer produces a beam pattern of high frequency. One transducer produces a high frequency that is higher by a predetermined quantity than the other. At their point of intersection, the sonic oscillations add and subtract producing a low frequency beat equal to the predetermined quantity. This high energy, low frequency beat can be used to stimulate neural points in the skull or other parts of the body or for tissue destruction. In a related method, the high frequency beams are set in axial alignment so that the frequency generating output is fixed between the transducers. A master modulator can then be used to electronically vary the position of the intersecting locus along the axial line connecting the transducers.

(5) U.S. Pat. No. 4,674,505 to Pauli et al.

U.S. Pat. No. 4,674,505 issued to Pauli et al. on Jun. 23, 1987 in U.S. class 601 and subclass 4 teaches an essentially planar shock wave generated with the assistance of a shock wave tube via a magnetic dynamic effect. The shock wave is focused by an acoustic convergent lens, whereby the calculus to be pulverized is placed at the focal point of the convergent lens. In order to couple the shock wave to the patient, the space that the shock wave traverses is filled with a coupling agent, for example, water. The shock wave tube, the convergent lens, and a fine adjustment for the displacement of the convergent lens relative to the shock wave tube are attached to a mounting stand so as to be pivotable in all directions. The disintegration facility includes a shock wave tube having high operating reliability with respect to high voltage, requires low maintenance, and has only negligible imaging or focusing errors resulting from the shock wave producing membrane and the convergent lens.

(6) U.S. Pat. No. 4,747,142 to Tofte.

U.S. Pat. No. 4,747,142 issued to Tofte on May 24, 1988 in U.S. class 381 and subclass 27 teaches a stereophonic reproduction system. A true center-channel signal is derived by combining the left and right signals into a monophonic signal, and canceling and overriding this monophonic signal with a second modified monophonic signal. The latter is derived by combining properly bandpassed left and right signals that have been compressed, combined, and expanded. True left- and true right-channel signals are subsequently derived by subtracting the true center-channel signal voltage from the left and right signal voltages.

(7) U.S. Pat. No. 4,753,225 to Vogel.

U.S. Pat. No. 4,753,225 issued to Vogel on Jun. 28, 1988 in U.S. class 601 and subclass 47 teaches therapy equipment for the human body serving to enhance the feeling of good health by exposure of a part of or all of the body to acoustic irradiation with frequencies in the sub-audio, audio, and ultrasonic regions. The therapy equipment includes at least one oscillator plate arranged in bodily contact with the body of the person who sits, lies, or stands on it. The oscillator plate is made to oscillate by sound waves, whereby corresponding oscillation generators are secured in bodily contact to the oscillator plate. The frequency of the sound waves is adjusted to the reabsorption frequency of individually selected organs and parts of the body to treat selective individual organs or parts of the human body.

(8) U.S. Pat. No. 5,062,412 to Okazaki.

U.S. Pat. No. 5,062,412 issued to Okazaki on Nov. 5, 1991 in U.S. class 601 and subclass 4 teaches electrically, and simultaneously, forming a plurality of focused regions of shock waves. A shock wave generating apparatus includes a plurality of high-voltage pulse generators for generating a plurality of high-voltage pulses, a shock wave generating unit having a plurality of ultrasonic vibrating element groups coupled to the plurality of high voltage pulse generators for generating shock waves and for focusing the shock waves onto a plurality of different focused regions within a biological body under examination, and a plurality of delay units coupled via the high-voltage pulse generators to the plural ultrasonic vibrating element groups for causing the plurality of high-voltage pulses having predetermined delay times to be generated from the high-voltage pulse generators, whereby the plural focused regions are simultaneously formed juxtaposed each other near a concretion to be disintegrated with the biological body.

(9) U.S. Pat. No. 5,086,755 to Schmid-Eilber.

U.S. Pat. No. 5,086,755 issued to Schmid-Eilber on Feb. 11, 1992 in U.S. class 601 and subclass 47 teaches a chaise lounge for therapeutic treatment of a patient, which includes three support sections hinged together so as to be pivotable relative to one another for comfortably supporting a patient. The support sections have openings formed therein that are spaced along the longitudinal centerline of the chaise lounge and electroacoustic transducers movably disposed below the openings and adapted to radiate upwardly through the openings at the lower back, the chest, and the head/neck areas of a patient resting on the chaise longue with an enhanced signal of a frequency corresponding to the rhythm frequency of certain music to which the patient's body is exposed. The rhythm frequency is in the non-audible range and adapted to achieve total relaxation of the patient.

(10) U.S. Pat. No. 5,095,890 to Houghton et al.

U.S. Pat. No. 5,095,890 issued to Houghton et al. on Mar. 17, 1992 in U.S. class 601 and subclass 2 teaches a method for automatically optimizing ultrasonic frequency power applied by a transducer to human tissue while the transducer is energized with ultrasonic signals from an ultrasonic signal generator. The frequency of an ultrasonic energizing signal applied by the ultrasonic signal generator to the transducer is set. The frequency of the energizing signal applied to the ultrasonic signal generator to the transducer is scanned at reoccurring intervals through a sequence of frequencies. The optimum level of power from the transducer is monitored as the frequency is scanned. The frequency of the ultrasonic energizing signal applied by the ultrasonic signal generator is ultimately reset substantially at the frequency that causes the optimum level of power until the next reoccurring interval.

(11) U.S. Pat. No. 5,143,063 to Fellner.

U.S. Pat. No. 5,143,063 issued to Fellner on Sep. 1, 1992 in U.S. class 601 and subclass 2 teaches electromedical apparatus employed to non-invasively remove adipose tissue from the body by causing necrosis thereof by localizing, e.g., focusing, radiant energy. The radiant energy may be of any suitable kind, e.g., localized radio frequency, microwave, or ultrasound energy, that is impinged upon the cells to be eliminated. Cell destruction occurs through a mechanism, such as, e.g., heating or mechanical disruption beyond a level that the adipose tissue can survive.

(12) U.S. Pat. No. 5,144,953 to Wurster et al.

U.S. Pat. No. 5,144,953 issued to Wurster et al. on Sep. 8, 1992 in U.S. class 600 and subclass 439 teaches a lithotritor having an X-ray alignment system that includes a transducer for generating focused ultrasonic shock waves adapted for alignment on a concretion or tissue to be destroyed. The transducer is connected to an image-forming diagnostic X-ray system for locating the concretion or tissue, and includes an X-ray emitter and an image intensifier disposed on a pivotable frame. The transducer is connected to the X-ray emitter that in turn is disposed at the center of the transducer so that the emission axes of the transducer and the X-ray emitter coincide.

(13) U.S. Pat. No. 5,178,134 to Vago.

U.S. Pat. No. 5,178,134 issued to Vago on Jan. 12, 1993 in U.S. class 601 and subclass 2 teaches ultrasonic treatment of animals. Ultrasonic waves in a frequency range of between 15 kilohertz and 100 kilohertz are applied to water in a tube with a power density between 0.1 and 5 watts per square centimeter. The equipment is able to apply ultrasonic waves with at least two power densities in the vicinity of the portion of the animal with one of the power densities being more than 15 watts per square meter for sterilizing the water before the patient enters the tube and the other being less than 15 watts per square meter.

(14) U.S. Reissue Pat. No. 34,219 to Lederer.

U.S. Reissue Pat. No. 34,219 reissued to Lederer on Apr. 13, 1993 in U.S. class 381 and subclass 334 teaches a sound or acoustic system constructed entirely of magnetically inert components that remain unaffected by magnetic fields. A magnetically inert panel is provided, with at least one magnetically-inert transducer being mounted upon the panel. At least one hollow channel extends into the panel from an edge thereof, while a hole is positioned within the panel to communicate at one end with the channel and to open at an opposite end adjacent the transducer.

(15) U.S. Pat. No. 5,209,221 to Riedlinger.

U.S. Pat. No. 5,209,221 issued to Riedlinger on May 11, 1993 in U.S. class 601 and subclass 2 teaches a device for generating sonic signal forms for limiting, preventing, or regressing the growth of pathological tissue, which includes an ultrasonic transmission system for transmitting sound waves focused on the tissue to be treated by way of a coupling medium. An ultrasonic signal, produced at the focus of the system, includes brief pulses having at least one rarefaction phase with a negative sonic pressure amplitude having a value greater than 2×10⁵ Pa. The ultrasonic signal is radiated with a carrier frequency exceeding 20 kHz, a sonic pulse duration T of less than 100 mus, and a pulse recurrence rate of less than 1/(5T). The device produces controlled cavitation in the tissue to be treated.

(16) U.S. Pat. No. 5,222,484 to Krauss et al.

U.S. Pat. No. 5,222,484 issued to Krauss et al. on Jun. 29, 1993 in U.S. class 601 and subclass 4 teaches an apparatus for shock wave treatment, which includes a shock wave transducer with a cup-shaped body and an X-ray location finding device for finding the location of a bodily concretion or tissue to be treated. The X-ray device includes an extendable X-ray tube with telescoping tube sections that are sealed against an acoustic coupling medium filling the delay path of the transducer by a balloon filling arranged within the X-ray tube. The balloon is secured to the upper section of the tube and to the lower section thereof. Over pressure or under pressure is applied to the interior of the X-ray tube to adjust its length in order to optimize X-ray location finding on the one hand, and shock wave treatment on the other hand.

(17) U.S. Pat. No. 5,388,581 to Bauer et al.

U.S. Pat. No. 5,388,581 issued to Bauer et al. on Feb. 14, 1995 in U.S. class 600 and subclass 427 teaches a therapy apparatus for treating concretions and tissue in the body of a patient by way of sound waves. The apparatus includes a sound wave generator and an available X-ray device for locating an object for therapy. The therapy apparatus has a spot film device arranged within the axial passage of an X-ray cone. The available X-ray device is attached to the sound wave generator, with its central longitudinal axis aligned with the focus thereof so as to be able to precisely adjust and fix the X-ray device to the therapy apparatus.

(18) U.S. Pat. No. 5,413,550 to Castel

U.S. Pat. No. 5,413,550 issued to Castel on May 9, 1995 in U.S. class 601 and subclass 2 teaches an ultrasound therapy system with automatic dose control, which includes an ultrasound transducer, an ultrasound generator controllable in frequency and power output connected to the transducer, a system controller interfaced to the generator, input switches interfaced to the controller to enable the input of selected ultrasound treatment parameters, and a display interfaced to the controller. The controller is programmed to calculate an ultrasound treatment dosage in terms of frequency, ultrasound intensity, and treatment time from the entered treatment parameters. Once treatment is started, the controller tracks the accumulated dosage applied to the tissue. The clinician can vary the intensity or treatment time, and the controller will recalculate the other factor for the remaining portion of the unapplied treatment dosage.

(19) U.S. Pat. No. 5,435,311 to Umemura et al.

U.S. Pat. No. 5,435,311 issued to Umemura et al. on Jul. 25, 1995 in U.S. class 600 and subclass 439 teaches an ultrasound therapeutic system provided with an ultrasound transmitter having a focusing mechanism, and a plurality of groups of ultrasound transmitters/receivers, each of which has a controllable directivity. Each of the transmitters/receivers is constructed so as to be able to receive both echo of pulse-shaped ultrasound transmitted by itself and even order harmonic signals of the ultrasound transmitted by the transmitter. A plurality of two-dimensional pulse echographical images are constructed by ultrasound signals obtained by transmitting/receiving beams while controlling the directivity of the beam emitted by each of the plurality of groups of ultrasound transmitters/receivers and a plurality of images indicating orientation and intensity, in which an even order harmonic wave signal due to the ultrasound transmitted by the transmitter is received by each of the plurality of groups of ultrasound transmitters/receivers, are displayed, superimposed on each other.

(20) U.S. Pat. No. 5,388,581 to Bauer et al.

U.S. Pat. No. 5,388,581 issued to Bauer et al. on Feb. 14, 1995 in U.S. class 600 and subclass 427 teaches a therapy apparatus for treating concretions and tissue in the body of a patient by way of sound waves. The apparatus includes a sound wave generator and an available X-ray device for locating an object for therapy. The therapy apparatus has a spot film device that is arranged within the axial passage of an X-ray cone. The available X-ray device is attached to the sound wave generator, with its central longitudinal axis aligned with the focus thereof so as to be able to adjust and fix the X-ray device to the therapy apparatus.

(21) U.S. Pat. No. 5,498,236 to Dubrul et al.

U.S. Pat. No. 5,498,236 issued to Dubrul et al. on Mar. 12, 1996 in U.S. class 604 and subclass 22 teaches a catheter suitable for introduction into a tubular tissue for dissolving blockages in the tissue. The catheter is particularly useful for removing thrombi within blood vessels. In accordance with the preferred embodiments, a combination of vibrating motion and injection of a lysing agent is utilized to break up blockages in vessels. The vessels may be veins, arteries, ducts, intestines, or any lumen within the body that may become blocked from the material that flows through it. As a particular example, dissolution of vascular thrombi is facilitated by advancing a catheter through the occluded vessel, with the catheter causing a vibrating stirring action in and around the thrombus usually in combination with the dispensing of a thrombotic agent, such as urokinase, into the thrombus. The catheter has an inflatable or expandable member near the distal tip thereof, which when inflated or expanded prevents the passage of dislodged thrombus around the catheter. The dislodged portions of thrombus are directed through a perfusion channel in the catheter where they are removed by filtration apparatus housed within the perfusion channel before the blood exits the tip of the catheter. Catheters that allow both low frequency—1-1000 Hz—vibratory motion and delivery of these agents to a blockage and a method for using the catheters are further taught.

(22) U.S. Pat. No. 5,501,655 to Rolt et al.

U.S. Pat. No. 5,501,655 to issued Rolt et al. on Mar. 26, 1996 in U.S. class 601 and subclass 3 teaches an ultrasound hyperthermia applicator suitable for medical hyperthermia treatment, and method a for using it. The applicator includes two ultrasound sources producing focused ultrasound beams of frequencies f₀ and f₁. An aiming device directs the two ultrasound beams so that they cross each other confocally at the target. A controller activates the two ultrasound sources so that the target is simultaneously irradiated by the two focused ultrasound beams. The two ultrasound sources provide acoustic energy sufficient to cause sufficient intermodulation products to be produced at the target as a result of the interaction of the two ultrasound beams. The intermodulation products are absorbed by the target to enhance heating of the target. In preferred embodiments, the ultrasound sources include a pair of signal generators for producing gated ultrasound output signals driving single crystal ultrasound transducers. In other embodiments, the ultrasound sources include a pair of phased array ultrasound transducers for generating two separate ultrasound beams. An aiming device is provided for electronically steering and focusing the two ultrasound beams so that they cross each other confocally at the target. Further embodiments employ pluralities of transducers, arrays, or both.

(23) U.S. Pat. No. 5,503,150 to Evans.

U.S. Pat. No. 5,503,150 issued to Evans on Apr. 2, 1996 in U.S. class 600 and subclass 427 teaches a method and apparatus for noninvasively locating and heating a volume of tissue, specifically a cancerous tumor. The method includes placing a bolus in contact with the patient and substantially around an area of interest including the volume of tissue, placing an array of antennas on the bolus and substantially around the area of interest, imaging the area of interest, selecting an approximate center of the volume of tissue on the initial image, determining approximate amplitudes and phases for the antennas, energizing each element at respective appropriate amplitudes and phases to heat the volume of tissue, imaging respectively the area of interest to create subsequent images, and subtracting the initial image from the subsequent images to determine temperature changes in the area of interest.

(24) U.S. Pat. No. 5,524,625 to Okazaki et al.

U.S. Pat. No. 5,524,625 issued to Okazaki et al. on Jun. 11, 1996 in U.S. class 600 and subclass 439 teaches a shock wave generating system capable a forming a wide concretion-disintegrating region by energizing ring-shaped transducers and a hyperthermia curing system. A width of a focused region synthesized from a plurality of focal points formed by a plurality of shock waves is varied by properly controlling delay times and/or drive voltages for a plurality of ring-shaped piezoelectric transducer elements. The shock wave generating system includes a shock wave generating unit having a plurality of shock wave generating elements and a driving unit for separately driving the plurality of shock wave generating elements by controlling at least delay times to produce a plurality of shock waves in such a manner that a dimension of a focused region synthesized from a plurality of different focal points formed by the plurality of shock waves is varied in accordance with a dimension of a concretion to be disintegrated that is present in a biological body under medical examination.

(25) U.S. Pat. No. 5,529,572 to Spector.

U.S. Pat. No. 5,529,572 issued to Spector on Jun. 25, 1996 in U.S. class 601 and subclass 2 teaches a method and apparatus for increasing the density and strength of bone, particularly for preventing or treating osteoporosis, by subjecting the bone to unfocused compressional shock waves.

(26) U.S. Pat. No. 5,542,906 to Herrman et al.

U.S. Pat. No. 5,542,906 issued to Herrman et al. on Aug. 6, 1996 in U.S. class 601 and subclass 2 teaches a therapy apparatus that has a source of acoustic waves that generates acoustic waves focused onto a focus, and an X-ray locating apparatus with which the subject to be treated can be irradiated from different directions. The central ray of the locating apparatus assumes a first direction for a first irradiation direction and a second direction for a second irradiation direction. The apparatus has a positioning system with which the subject to be treated and the focus can be adjusted relative to one another. The region to be treated and the focus are adjustable relative to one another by synchronous actuation of the positioning system in two adjustment directions for at least one irradiation direction. The adjustment takes place in a direction that proceeds parallel to the direction of the central ray that belongs to the other irradiation direction.

(27) U.S. Pat. No. 5,549,544 to Young et al.

U.S. Pat. No. 5,549,544 issued to Young et al. on Aug. 27, 1996 in U.S. class 601 and subclass 2 teaches an apparatus including a piezoelectric vibrator adapted to generate ultrasonic energy that is transmitted through an output section to a plastic head. The shape of the head may be varied to suit whichever part of a body on which it is to be used. The material and shape of the head is chosen to allow accurate control of frequency and amplitude of the ultrasonic energy. The preferred ultrasonic frequency is in the range of 20-120 kHz.

(28) U.S. Pat. No. 5,558,623 to Cody.

U.S. Pat. No. 5,558,623 issued to Cody on Sep. 24, 1996 in U.S. class 601 and subclass 2 teaches a therapeutic ultrasonic device that transmits multiple ultrasonic frequencies through one ultrasonic applicator. The applicator includes a handle, two diaphragms connected to one end of the handle, with each diaphragm having an applicating face directed away from the handle and a rear face directed into the handle so that the applicating faces may be independently applied to a patient during therapy, and at least two piezoelectric crystals. A piezoelectric crystal is connected to the rear face of each diaphragm for converting periodic electrical energy into ultrasonic energy and transmitting the ultrasonic energy through the diaphragm to which the crystal is connected independently of the other diaphragm. An excitation source is provided for independently applying a periodic electric field of selectable frequency across a crystal in order to select the crystal to receive the periodic electric field and to select the ultrasonic frequency transmitted through the diaphragm to which the selected crystal is connected.

(29) U.S. Pat. No. 5,713,848 to Dubrul et al.

U.S. Pat. No. 5,713,848 issued to Dubrul et al. on Feb. 3, 1998 in U.S. class 604 and subclass 22 teaches a catheter suitable for introduction into a tubular tissue for dissolving blockages in the tissue. The catheter is particularly useful for removing thrombi within blood vessels. In accordance with the preferred embodiments, a combination of vibrating motion and injection of a lysing agent is utilized to break up blockage in vessels. The vessels may be veins, arteries, ducts, intestines, or any lumen within the body that may become blocked from the material that flows through it. As a particular example, dissolution of vascular thrombi is facilitated by advancing a catheter through the occluded vessel. The catheter causes a vibrating stirring action in and around the thrombus usually in combination with the dispensing of a thrombolytic agent, such as urokinase into the thrombus. The catheter has an inflatable or expandable member near the distal tip thereof that when inflated or expanded prevents the passage of dislodged thrombus around the catheter. The dislodged portions of thrombus are directed through a profusion channel in the catheter where they are removed by filtration apparatus housed within the perfusion channel before the blood exits the tip of the catheter. Catheters that allow both low frequency, i.e., 1-1000 Hz, vibratory motion and delivery of the agents to a blockage and a method for using the catheters are taught.

### (30) United States Patent Application Publication Number US 2001/0055812 A1 to Mian et al.

United States Patent Application Publication Number US 2001/0055812 A1 published to Mian et al. on Dec. 27, 2001 in U.S. class 436 and subclass 45 teaches methods and apparatus for performing microanalytic and microsynthetic analyses and procedures, which provides a microsystem platform and a micromanipulation device for manipulating the platform that utilizes the centripetal force resulting from rotation of the platform to motivate fluid movement through microchannels. The microsystem platforms are also provided with system informatics and data acquisition, and analysis and storage and retrieval informatics encoded on the surface of the disk opposite to the surface containing the fluidic components. Methods specific for the apparatus for performing any of a wide variety of microanalytical or microsynthetic processes are taught.

(31) U.S. Pat. No. 7,416,535 B1 to Kenny.

U.S. Pat. No. 7,416,535 B1 issued to Kenny on Aug. 26, 2008 in U.S. class 601 and subclass 2 teaches a neoplasm cell destruction device utilizing low frequency sound waves to disrupt or displace cellular materials in neoplastic cells so as to damage and ultimately destruct the neoplastic cells without destructing surrounding healthy cells and thereby eliminating the need for target finding apparatus. The device includes a plurality of signal generators, a controller, a plurality of amplifiers, a plurality of transducers, and a target interface. The controller is in electrical communication with, and generates timing and control signals for simultaneously activating, the plurality of signal generators. Each amplifier is in electrical communication with a respective signal generator and amplifies the signal generated by the respective signal generator so as to form an amplified signal. Each transducer is in electrical communication with a respective amplifier and is driven by the amplified signal formed by the respective amplifier. The target interface combines the waveforms formed by the plurality of transducers to form an interference wave that is a low frequency sound wave.

It is apparent that numerous innovations for wave-related devices have been provided in the prior art, which are adapted to be used. Furthermore, even though these innovations may be suitable for the specific individual purposes to which they address, however, they would not be suitable for the purposes of the embodiments of the present invention as heretofore described, namely, a cancer cell destruction device utilizing low frequency sound waves to disrupt or displace cellular materials in cancerous cells.

### 3. SUMMARY OF THE INVENTION

Thus, an object of the embodiments of the present invention is to provide a neoplasm cell destruction device utilizing low frequency sound waves to disrupt or displace cellular materials, which avoids the disadvantages of the prior art.

Neoplastic cells trade in their ability to heal themselves in return for uncontrollable reproduction. If the neoplasm cells per se became damaged, they could therefore not heal themselves and they would therefore eventually be destroyed.

Briefly stated, another object of the embodiments of the present invention is to provide a neoplasm cell destruction device utilizing low frequency sound waves to disrupt or displace cellular materials in neoplastic cells having resonant frequencies so as to damage and ultimately destruct the neoplastic cells. The device includes a plurality of signal generators, a controller, a plurality of amplifiers, a plurality of transducers, and a target interface. The controller is in electrical communication with, and generates timing and control signals for simultaneously activating, the plurality of signal generators. Each amplifier is in electrical communication with a respective signal generator and amplifies the signal generated by the respective signal generator so as to form an amplified signal. Each transducer is in electrical communication with a respective amplifier and is driven by the amplified signal formed by the respective amplifier. The target interface combines the waveforms formed by the plurality of transducers to form an interference wave that is a low frequency sound wave.

The novel features considered characteristic of the embodiments of the present invention are set forth in the appended claims. The embodiments of the present invention themselves, however, both as to their construction and their method of operation together with additional objects and advantages thereof will be best understood from the following description of the specific embodiments when read and understood in connection with the accompanying drawing.

### 4. BRIEF DESCRIPTION OF THE DRAWINGS

The figures on the drawing are briefly described as follows:

FIGS. 1A-1C are a block diagram of the embodiments of the present invention;

FIGS. 2A-2K are a flow chart of the embodiments of the present invention;

FIG. 3 is a diagrammatic perspective view of a first embodiment of the target interface utilized to treat the entire body of a patient;

FIG. 4 is a diagrammatic perspective view of a second embodiment of the target interface utilized to treat a small area of a patient;

FIG. 5 is a diagrammatic perspective view of a third embodiment of the target interface utilized to treat a large area of a patient; and

FIG. 6 is a diagrammatic perspective view of a fourth embodiment of the target interface utilized to treat a lumen of a patient.

### 5. DETAILED DESCRIPTION OF THE PREFERRED EMBODIMENTS

### A. General.

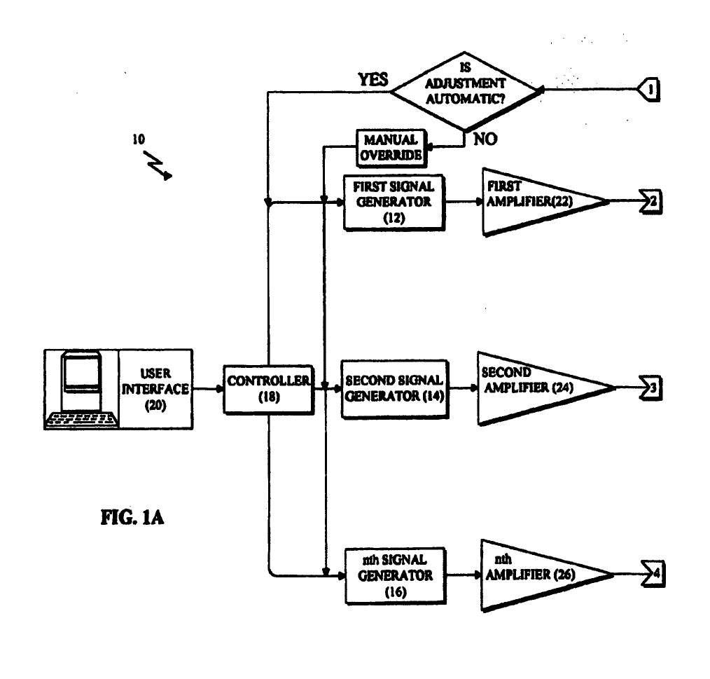

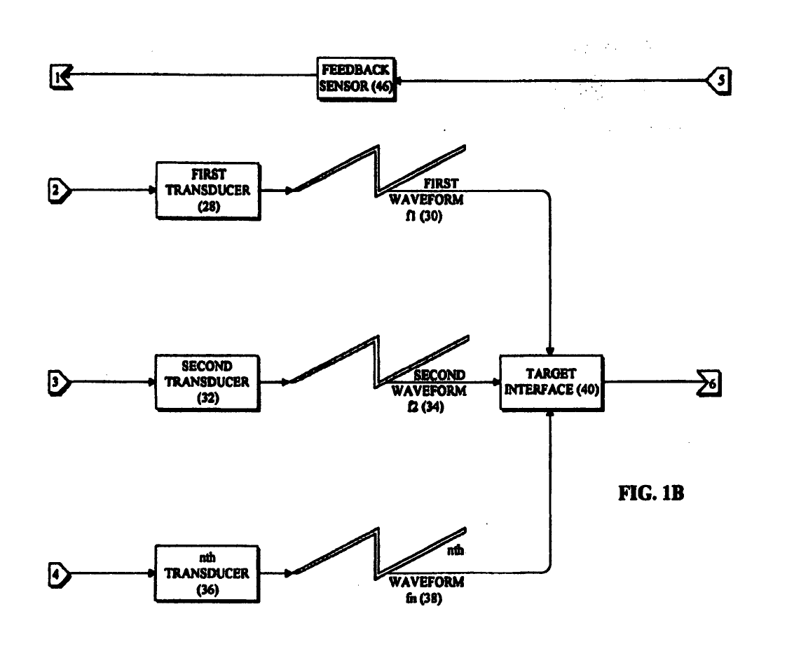

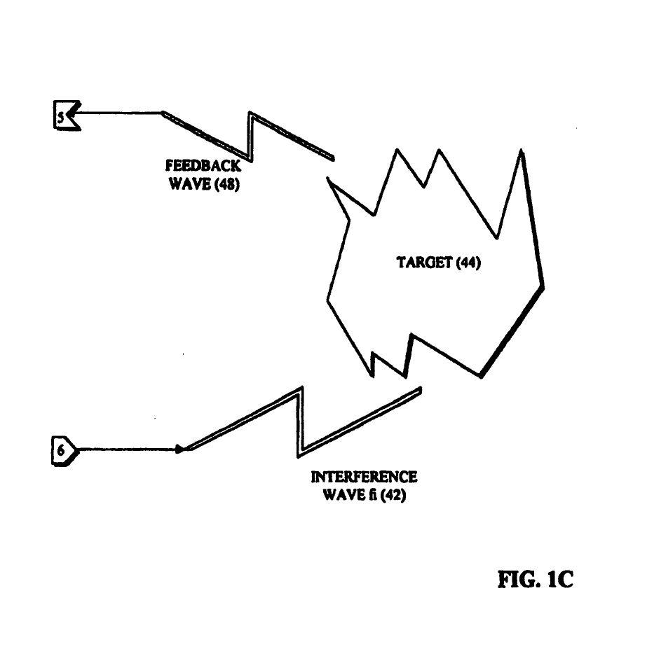

Referring now to the figures, in which like numerals indicate like parts, and particularly to FIGS. 1A-1C, which are a block diagram of the embodiments of the present invention, the neoplasm cell destruction device utilizing low frequency sound waves to disrupt or displace cellular materials of the embodiments of the present invention is shown generally at 10.

### B. The Configuration of the Neoplasm Call Destruction Device Utilizing Low Frequency Sound Waves to Disrupt or Displace Cellular Materials 10.

(1) The Plurality of Signal Generators 12, 14, and n^{th}.

The neoplasm call destruction device utilizing low frequency sound waves to disrupt or displace cellular materials 10 includes a plurality of signal generators.

The plurality of signal generators include a first signal generator 12. The first signal generator 12 generates a first signal at a frequency f₁ that is a low frequency sound wave.

The plurality of signal generators further includes a second signal generator 14. The second signal generator 14 generates a second signal at a frequency f₂ that is a low frequency sound wave.

The plurality of signal generators further include an n^{th} signal generator 16. The n^{th} signal generator 16 generates an n^{th} signal at an n^{th} frequency fₙ that is a low frequency sound wave, wherein n is any integer from 3 to ∞.

### (2) The Controller 18.

The neoplasm cell destruction device utilizing low frequency sound waves to disrupt or displace cellular materials 10 further includes a controller 18. The controller 18 is in electrical communication with, and generates timing and control signals to selectively activate, the first signal generator 12, the second signal generator 14, and the n^{th} signal generator 16.

The controller 18 can preferably be a microprocessor, but is not limited to that.

### (3) The User Interface 20.

The neoplasm cell destruction device utilizing low frequency sound waves to disrupt or displace cellular materials 10 further includes a user interface 20. The user interface 20 is in electrical communication with the controller 18.

The user interface 20 can preferably be a keyboard and a display or any other form thereof, but is not limited to that.

(4) The Plurality of Amplifiers 22, 24, and n^{th}.

The neoplasm cell destruction device utilizing low frequency sound waves to disrupt or displace cellular materials 10 further includes a plurality of amplifiers.

The plurality of amplifiers include a first amplifier 22. The first amplifier 22 is in electrical communication with the first signal generator 12, and amplifies the first signal generated thereby to form an amplified first signal.

The plurality of amplifiers further include a second amplifier 24. The second amplifier 24 is in electrical communication with the second signal generator 14, and amplifies the second signal generated thereby to form an amplified second signal.

The plurality of amplifiers further include an n^{th} amplifier 26. The n^{th} amplifier 26 is in electrical communication with the n^{th} signal generator 16, and amplifies the n^{th} signal generated thereby to form an amplified n^{th} signal, wherein n is an integer from 3 to ∞.

(5) The Plurality of Transducers 28, 32, and n^{th}.

The neoplasm cell destruction device utilizing low frequency sound waves to disrupt or displace cellular materials 10 further includes a plurality of transducers.

The plurality of transducers includes a first transducer 28. The first transducer 28 is in electrical communication with the first amplifier 22, and is driven by the amplified first signal to form a first waveform 30 that is a low frequency sound wave.

The plurality of transducers further includes a second transducer 32. The second transducer 32 is in electrical communication with the second amplifier 24, and is driven by the amplified second signal to form a second waveform 34 that is a low frequency sound wave.

The plurality of transducers further include an n^{th} transducer 36. The n^{th} transducer 36 is in electrical communication with the n^{th} amplifier 26, and is driven by the amplified n^{th} signal to form an n^{th} waveform 38 that is a low frequency sound wave, wherein n is any integer from 3 to ∞.

### (6) The Target Interface 40.

The neoplasm cell destruction device utilizing low frequency sound waves to disrupt or displace cellular materials 10 further includes a target interface 40. The target interface 40 combines the first waveform 30, the second waveform 34, and the n^{th} waveform 38 to form an interference wave 42 at a frequency fᵢ that is a low frequency sound wave. The interference wave 42 is impactable upon a neoplastic target 44 that has a resonant frequency, and damages the neoplastic target 44 by one of disrupting and displacing the cellular material of the neoplastic target 44, which leads to the ultimate death of the neoplastic target 44 by virtue of neoplasm cells trading in their ability to heal themselves in return for uncontrollable reproduction.

It is to be understood that the first waveform 30, the second waveform 34, and the n^{th} waveform 38 can preferably be different, and when combined, provide a synergistic effect in producing the interference wave 42, but is not limited to that.

### (7) The Feedback Sensor 46.

The neoplasm cell destruction device utilizing low frequency sound waves to disrupt or displace cellular materials 10 further includes a feedback sensor 46. The feedback sensor 46 is disposed in close proximity to the target interface 40, and is in electrical communication with the controller 18.

The feedback sensor 46 receives a feedback wave 48 emanating from the neoplastic target 44 when the neoplastic target 44 is impacted upon by the interference wave 42, and generates a feedback signal in response thereto that is received by the controller 18 that in turn continually compares the feedback signal to the interference wave 42 and automatically adjusts the first signal generator 12, the second signal generator 14, and the n^{th} signal generator 16 until the interference wave 42 is at the resonant frequency of the neoplastic target 44 so as to maximize damage to the neoplastic target 44.

It is to be understood that the controller 18 can be manually overridden and the feedback signal from the feedback sensor 46 would then go directly to the first signal generator 12, the second signal generator 14, and the n^{th} signal generator 16 that would be manually adjusted until the interference wave 42 is at the resonant frequency of the neoplastic target 44 so as to maximize damage to the neoplastic target 44.

### C. The Method of Operation of the Neoplasm Cell Destruction Device Utilizing Low Frequency Sound Waves to Disrupt or Displace Cellular Materials 10.

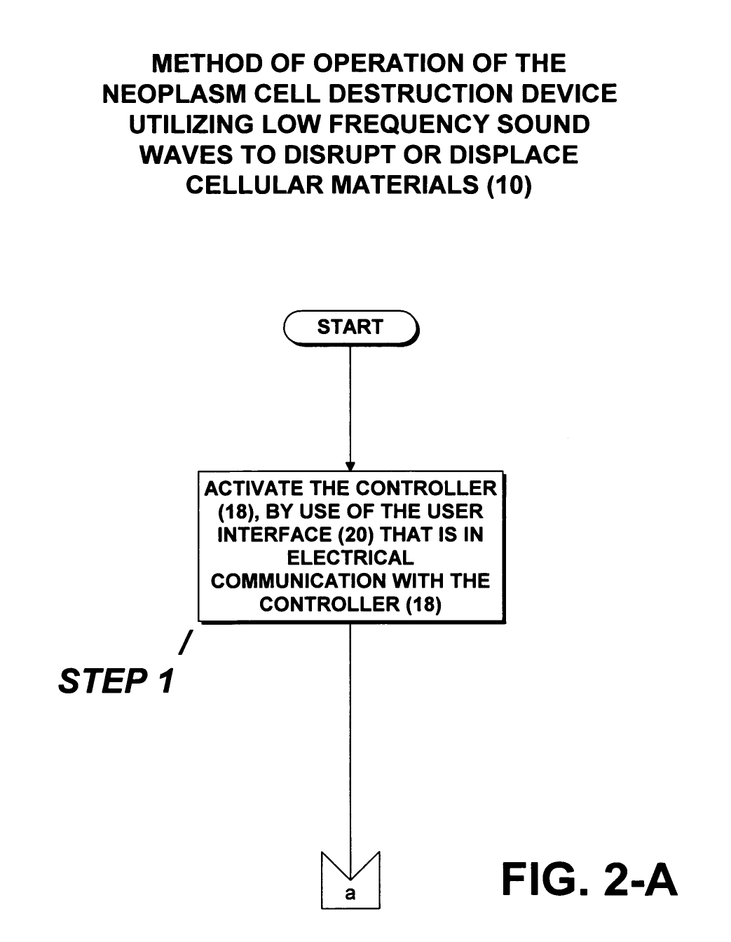

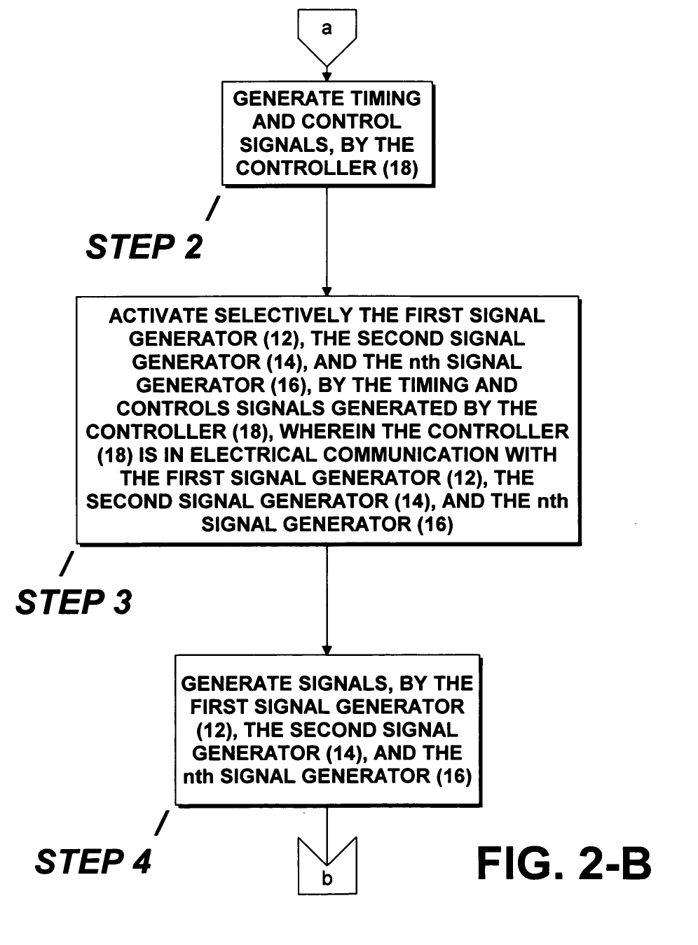

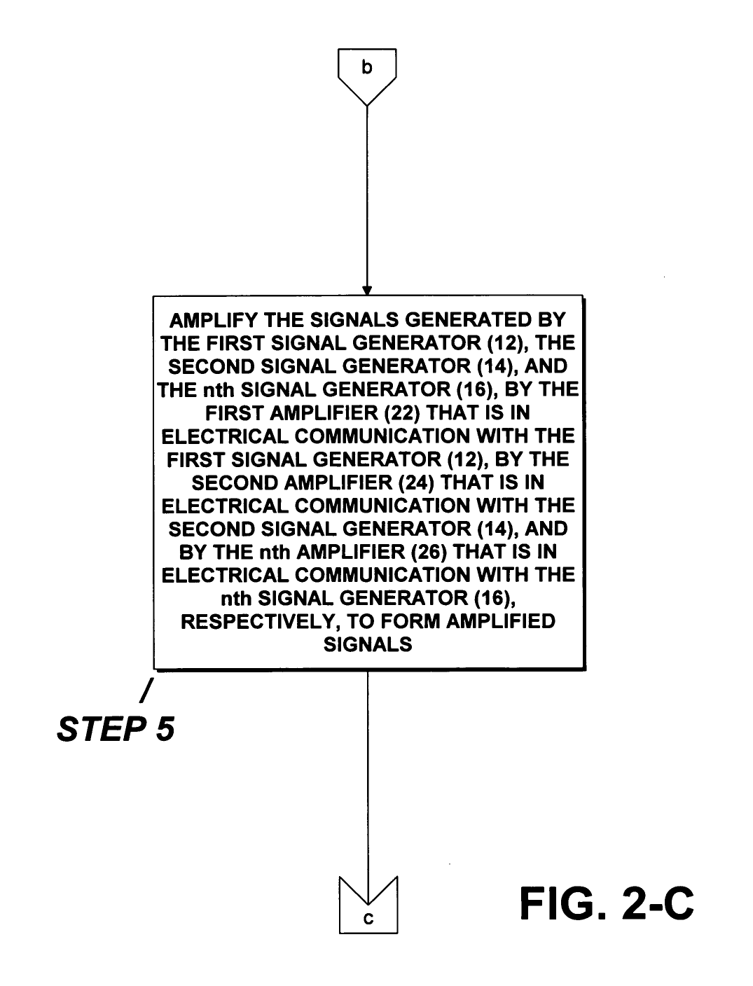

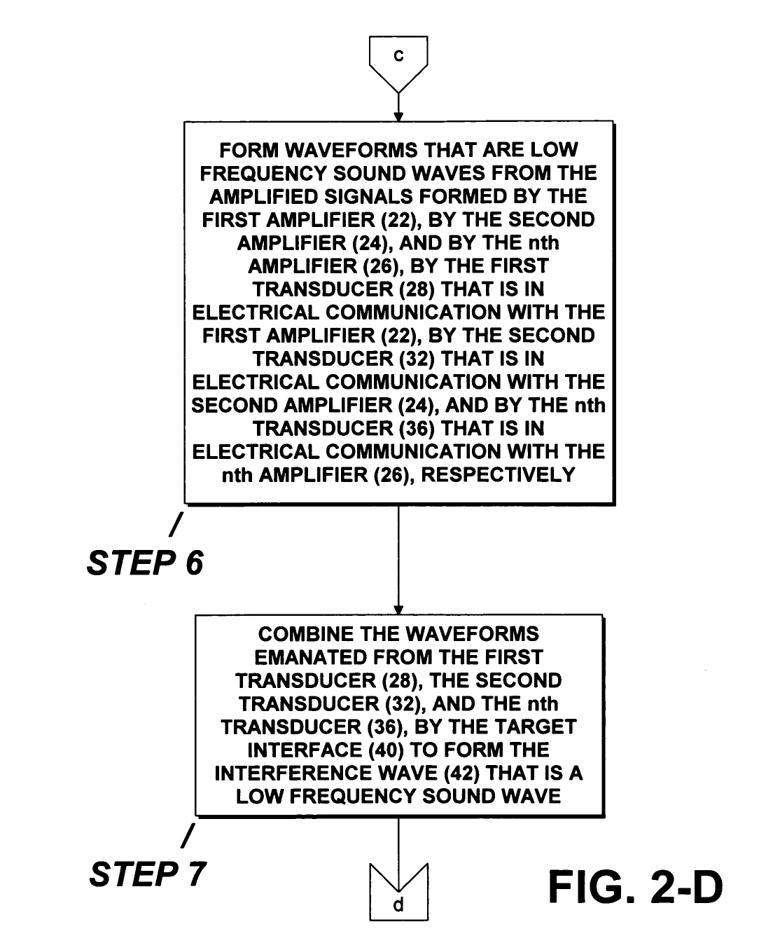

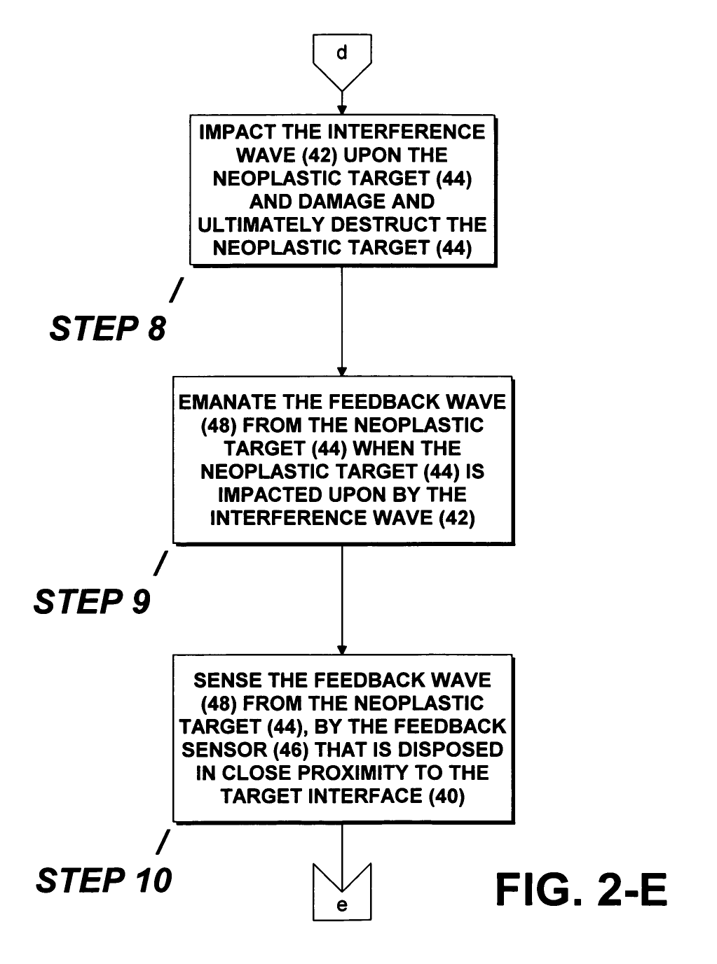

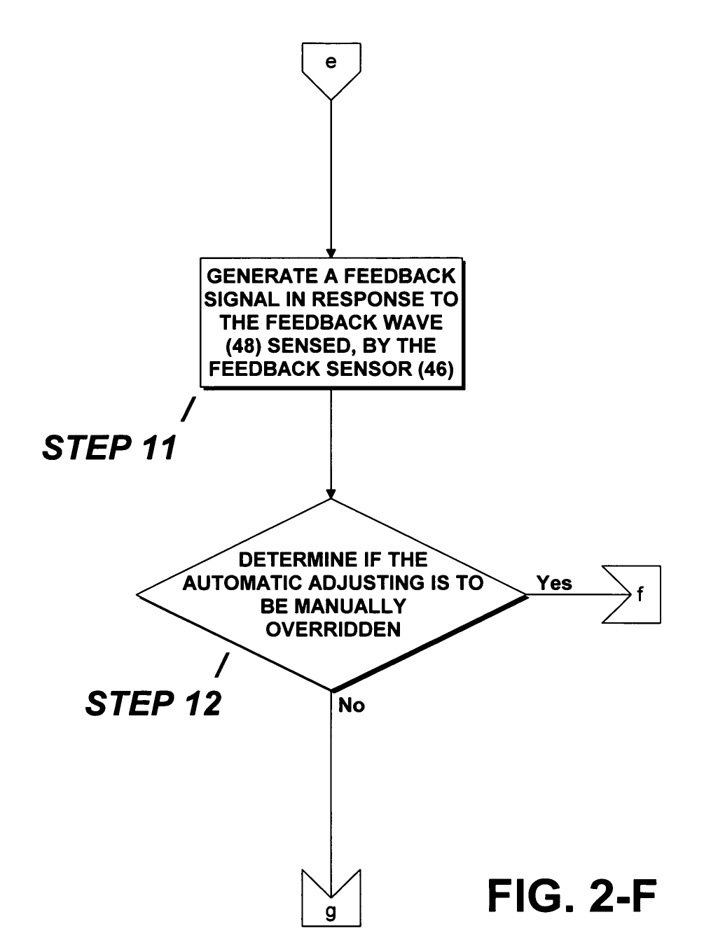

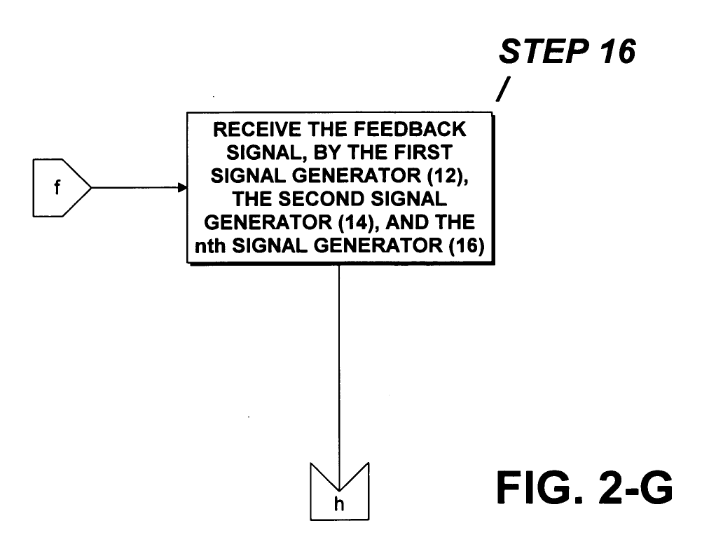

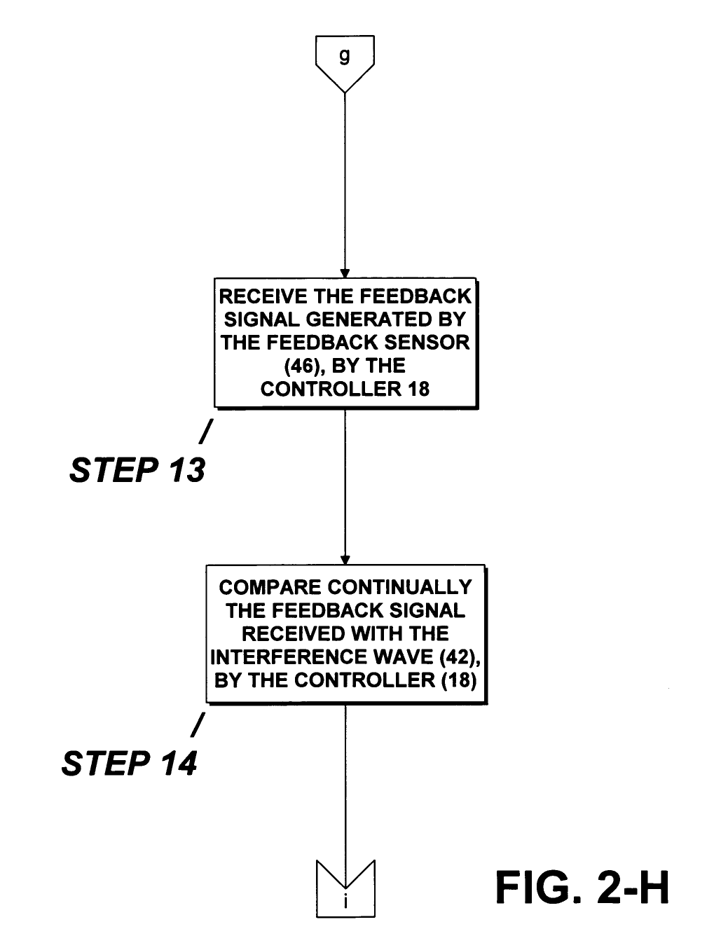

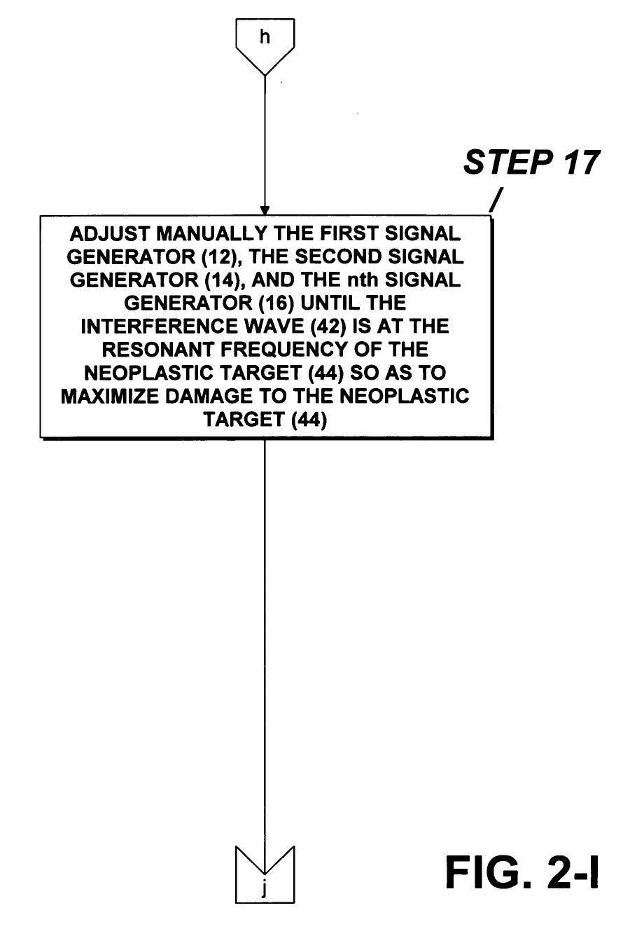

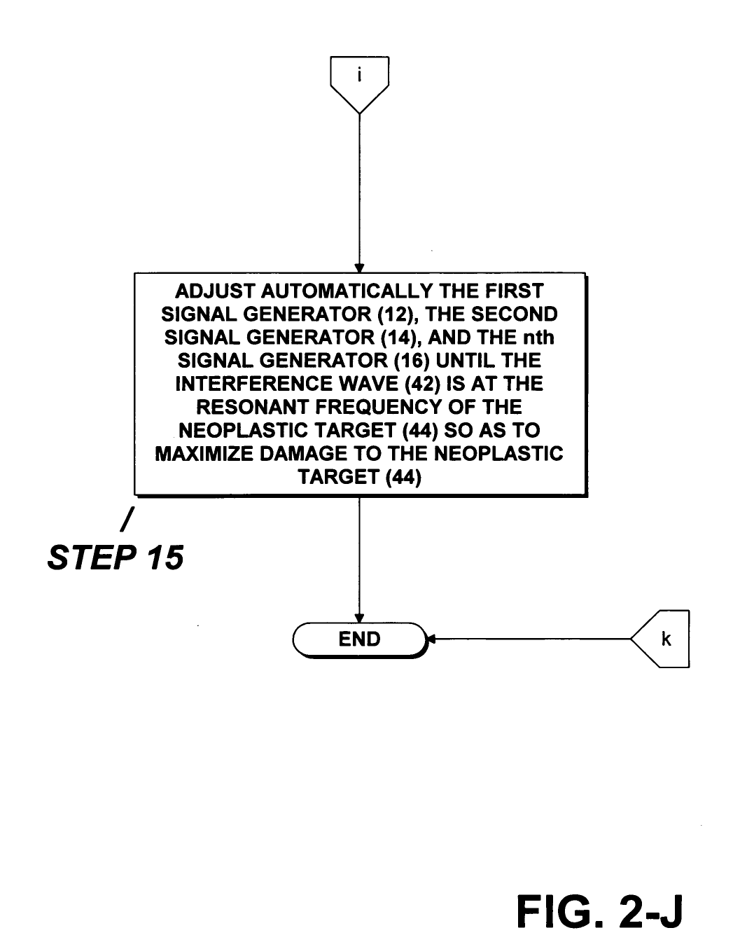

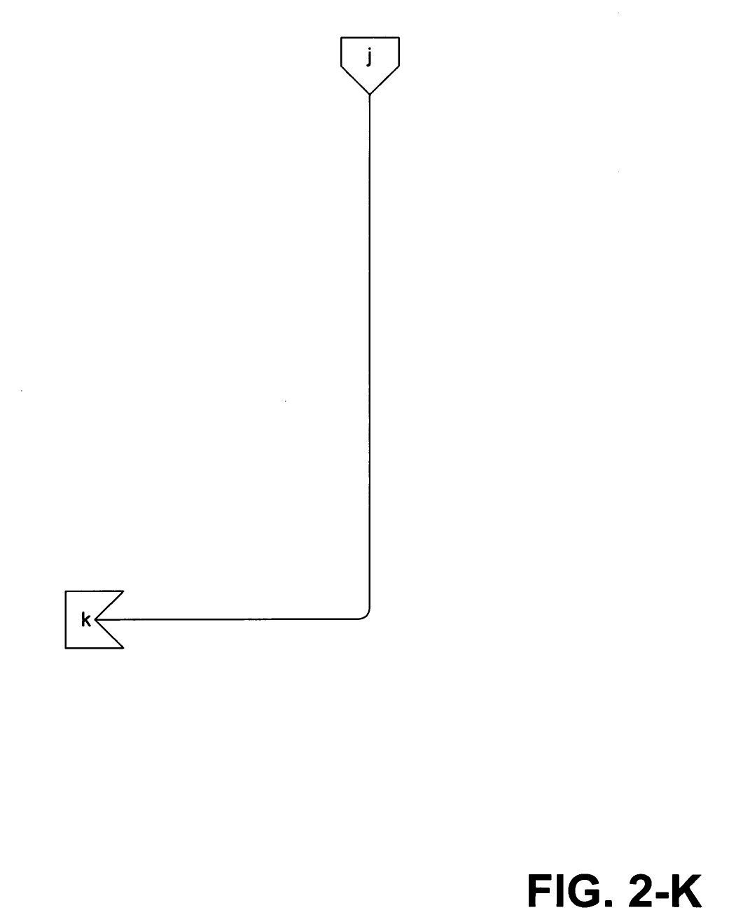

The method of operation of the neoplasm cell destruction device utilizing low frequency sound waves to disrupt or displace cellular materials 10 can best be seen in FIGS. 2A-2K, which are a flow chart of the embodiments of the present invention, and as such, will be discussed with reference thereto.
STEP 1: Activate the controller 18, by use of the user interface 20 that is in electrical communication with the controller 18.
STEP 2: Generate timing and control signals, by the controller 18.
STEP 3: Activate selectively the first signal generator 12, the second signal generator 14, and the n^{th} signal generator 16, by the timing and controls signals generated by the controller 18, wherein the controller 18 is in electrical communication with the first signal generator 12, the second signal generator 14, and the n signal generator 16.
STEP 4: Generate signals, by the first signal generator 12, the second signal generator 14, and the n signal generator 16.
STEP 5: Amplify the signals generated by the first signal generator 12, the second signal generator 14, and the n^{th} signal generator 16, by the first amplifier 22 that is in electrical communication with the first signal generator 12, by the second amplifier 24 that is in electrical communication with the second signal generator 14, and by the n^{th} amplifier 26 that is in electrical communication with the n^{th} signal generator 16, respectively, to form amplified signals.
STEP 6: Form waveforms that are low frequency sound waves from the amplified signals formed by the first amplifier 22, by the second amplifier 24, and by the n^{th} amplifier 26, by the first transducer 28 that is in electrical communication with the first amplifier 22, by the second transducer 32 that is in electrical communication with the second amplifier 24, and by the n^{th} transducer 36 that is in electrical communication with the n^{th} amplifier 26, respectively.
STEP 7: Combine the waveforms emanated from the first transducer 28, the second transducer 32, and the nth transducer 36, by the target interface 40 to form the interference wave 42 that is a low frequency sound wave.
STEP 8: Impact the interference wave 42 upon the neoplastic target 44 and damage and ultimately destruct the neoplastic target 44.
STEP 9: Emanate the feedback wave 48 from the neoplastic target 44 when the neoplastic target 44 is impacted upon by the interference wave 42.
STEP 10: Sense the feedback wave 48 from the neoplastic target 44, by the feedback sensor 46 that is disposed in close proximity to the target interface 40.
STEP 11: Generate a feedback signal in response to the feedback wave 48 sensed, by the feedback sensor 46.
STEP 12: Receive the feedback signal generated by the feedback sensor 46, by the controller 18.
STEP 13: Compare continually the feedback signal received with the interference wave 42, by the controller 18.
STEP 14: Adjust automatically the first signal generator 12, the second signal generator 14, and the n^{th} signal generator 16 until the interference wave 42 is at the resonant frequency of the neoplastic target 44 so as to maximize damage to the neoplastic target 44.

It is to be understood that the automatic adjusting that is accomplished by STEPS 12-14 can be manually overridden and replaced by the following manual steps:
STEP 12: Receive the feedback signal, by the first signal generator 12, the second signal generator 14, and the n^{th} signal generator 16.
STEP 13: Adjust manually the first signal generator 12, the second signal generator 14, and the n^{th} signal generator 16 until the interference wave 42 is at the resonant frequency of the neoplastic target 44 so as to maximize damage to the neoplastic target 44.

### D. The Configuration of a First Embodiment of the Target Interface 140.

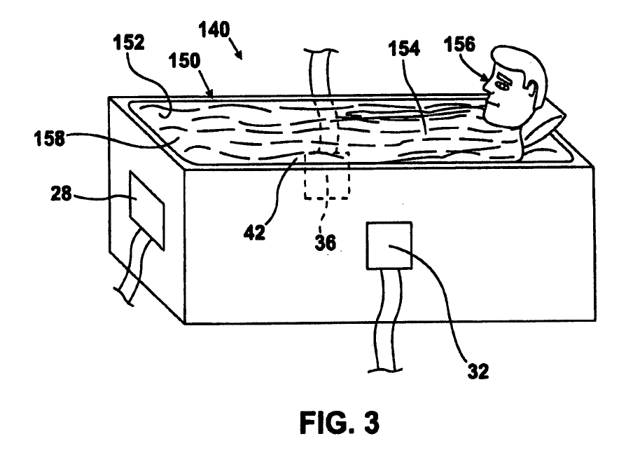

The configuration of a first embodiment of the target interface 140 can best be seen in FIG. 3, which is a diagrammatic perspective view of a first embodiment of the target interface utilized to treat the entire body of a patient, and as such will be discussed with reference thereto.

The target interface 140 includes a tub 150. The tub 150 of the target interface 140 defines an internal chamber 152 in which a body 154 of a patient 156 is placeable when the neoplastic target 44 is wide spread throughout the body 154 of the patient 156.

The first transducer 28, the second transducer 32, and the n^{th} transducer 36 are disposed on the tub 150 of the target interface 140, with the first waveform 30, the second waveform 34, and the n^{th} waveform 38 emanating therefrom into the internal chamber 152 in the tub 150 of the target interface 140.

The target interface 140 further includes a liquid 158. The liquid 158 of the target interface 140 is contained in the internal chamber 152 in the tub 150 of the target interface 140, communicates with the first transducer 28, the second transducer 32, the n^{th} transducer 36, and the body 154 of the patient 156, and functions as an acoustical coupler to combine the first waveform 30, the second waveform 34, and the n^{th} waveform 38 to form the interference wave 42.

### E. The Configuration of a Second Embodiment of the Target Interface 240.

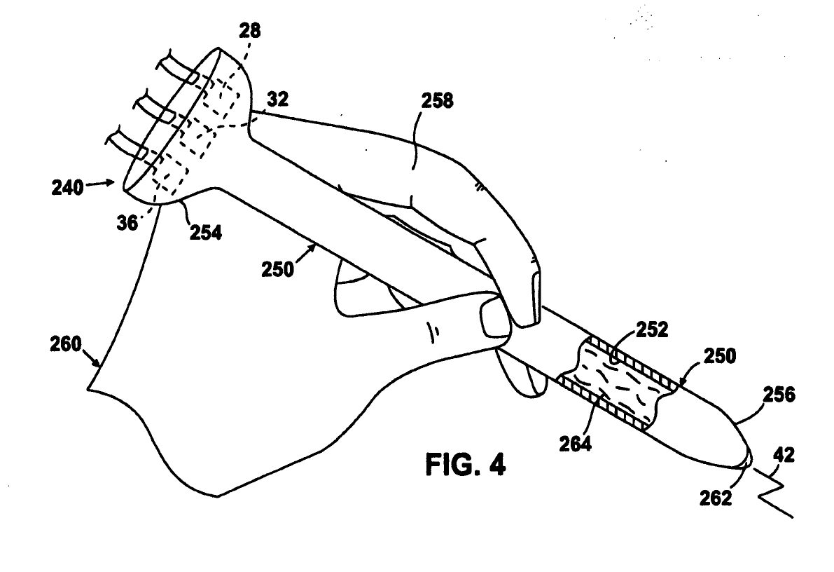

The configuration of a second embodiment of the target interface 240 can best be seen in FIG. 4, which is a diagrammatic perspective view of a second embodiment of the target interface utilized to treat a small area of a patient, and as such, will be discussed with reference thereto.

The target interface 240 includes a hollow, elongated, slender, hand-held, and tubular body 250. The hollow, elongated, slender, hand-held, and tubular body 250 of the target interface 240 contains an internal chamber 252, has a proximal end 254 and a distal treatment end 256, and is holdable in, and orientatably controllable by, a hand 258 of a user 260.

The hollow, elongated, slender, hand-held, and tubular body 250 of the target interface 240 can preferably be plastic and/or metal, but is not limited to that.

The first transducer 28, the second transducer 32, and the n^{th} transducer 36 are disposed at the proximal end 254 of the hollow, elongated, slender, hand-held, and tubular body 250 of the target interface 240, but is not limited to that, with the first waveform 30, the second waveform 34, and the n^{th} waveform 38 emanating therefrom into the internal chamber 252 in the hollow, elongated, slender, hand-held, and tubular body 250 of the target interface 240.

The target interface 240 further includes a diaphragm 262. The diaphragm 262 of the target interface 240 is disposed at, and closes, the distal treatment end 256 of the hollow, elongated, slender, hand-held, and tubular body 250 of the target interface 240, and concentrates the interference wave 42.

The diaphragm 262 of the target interface 240 communicates with the internal chamber 252 in the hollow, elongated, slender, hand-held, and tubular body 250 of the target interface 240, and is contactable at least in close proximity to the neoplastic target 44 when the neoplastic target 44 is contained in a small area in a body of a patient and pinpoint accuracy is required.

The target interface 240 further includes a liquid 264. The liquid 264 of the target interface 240 is contained in the internal chamber 252 in the hollow, elongated, slender, hand-held, and tubular body 250 of the target interface 240, communicates with the first transducer 28, the second transducer 32, the n^{th} transducer 36, and the diaphragm 262 of the target interface 240, and functions as an acoustical coupler to combine the first waveform 30, the second waveform 34, and the n^{th} waveform 38 to form the interference wave 42.

### F. The Configuration of a Third Embodiment of the Target Interface 340.

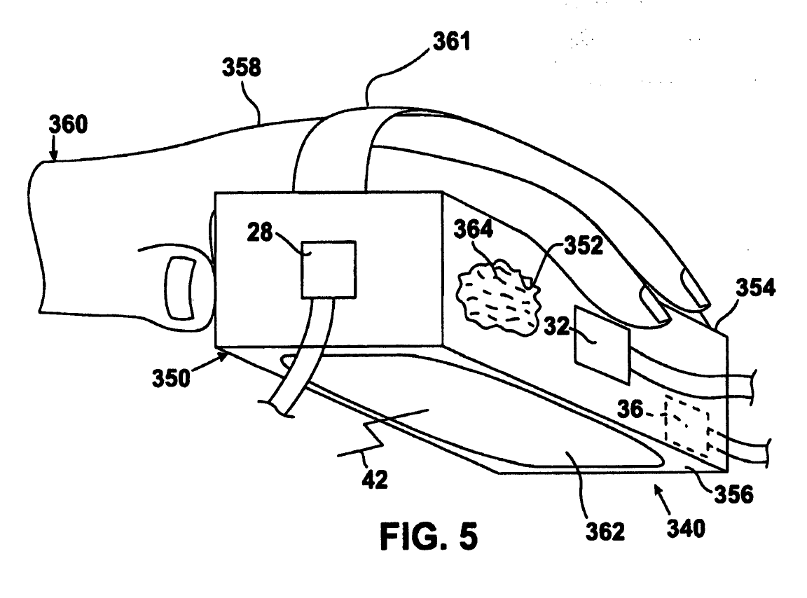

The configuration of a third embodiment of the target interface 340 can best be seen in FIG. 5, which is a diagrammatic perspective view of a third embodiment of the target interface utilized to treat a large area of a patient, and as such will be discussed with reference thereto.

The target interface 340 includes a hollow and hand-held body 350. The hollow and hand-held body 350 of the target interface 340 contains an internal chamber 352, has an upper wall 354 and a lower treatment wall 356, is holdable in a hand 358 of a user 360, and is contactable at least in close proximity to the neoplastic target 44 when the neoplastic target 44 is contained in a large area in a body of a patient.

The hand 358 of a user 360 is passable through a loop 361 of the target interface 340 that is accessible from the upper wall 354 of the hollow and hand-held body 350 of the target interface 340, but is not limited to that.

The hollow and hand-held body 350 of the target interface 340 can preferably be plastic and/or metal, but is not limited to that.

The first transducer 28, the second transducer 32, and the n^{th} transducer 36 are disposed at the hollow and hand-held body 350 of the target interface 340, with the first waveform 30, the second waveform 34, and the n^{th} waveform 38 emanating therefrom into the internal chamber 352 in the hollow and hand-held body 350 of the target interface 340.

The target interface 340 further includes a diaphragm 362. The diaphragm 362 of the target interface 340 is disposed at, and closes, the lower treatment wall 356 of the hollow and hand-held body 350 of the target interface 340, and distributes the interference wave 42.

The diaphragm 362 of the target interface 340 communicates with the internal chamber 352 in the hollow and hand-held body 350 of the target interface 340, and is contactable at least in close proximity to the neoplastic target 44 when the neoplastic target 44 is contained in a large area in a body of a patient.

The target interface 340 further includes a liquid 364. The liquid 364 of the target interface 340 is contained in the internal chamber 352 in the hollow and hand-held body 350 of the target interface 340, communicates with the first transducer 28, the second transducer 32, the n^{th} transducer 36, and the diaphragm 362 of the target interface 340, and functions as an acoustical coupler to combine the first waveform 30, the second waveform 34, and the n^{th} waveform 38 to form the interference wave 42.

### G. The Configuration of a Fourth Embodiment of the Target Interface 440.

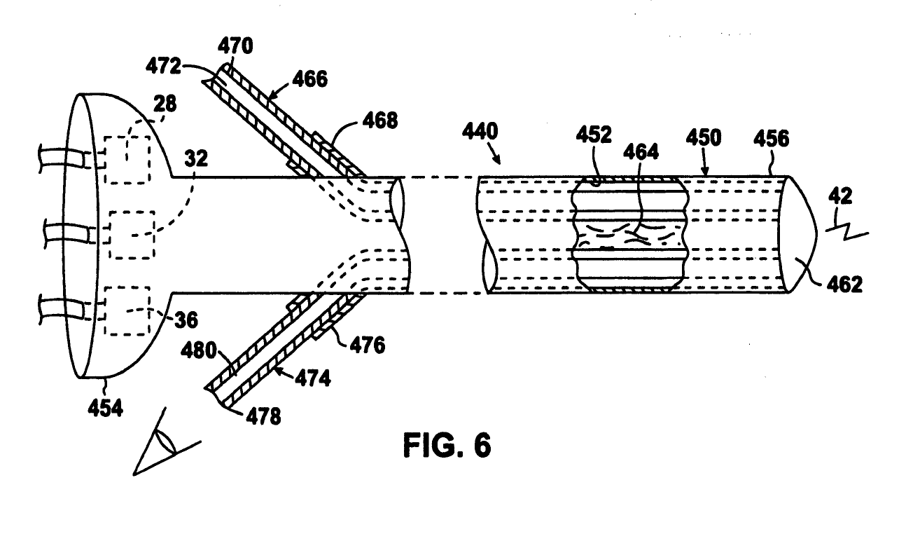

The configuration of a fourth embodiment of the target interface 440 can best be seen in FIG. 6, which is a diagrammatic perspective view of a fourth embodiment of the target interface utilized to treat a lumen of a patient, and as such, will be discussed with reference thereto.

The target interface 440 includes a catheter 450. The catheter 450 of the target interface 440 contains an internal chamber 452, has a proximal end 454 and a distal treatment end 456, and is contactable at least in close proximity to the neoplastic target 44 when the neoplastic target 44 is contained in a lumen in a body of a patient.

The first transducer 28, the second transducer 32, and the n^{th} transducer 36 are disposed at the proximal end 454 of the catheter 450 of the target interface 440, but is not limited to that, with the first waveform 30, the second waveform 34, and the n^{th} waveform 38 emanating therefrom into the internal chamber 452 in the catheter 450 of the target interface 440.

The target interface 440 further includes a diaphragm 462. The diaphragm 462 of the target interface 440 is disposed at, and closes, the distal treatment end 456 of the catheter 450 of the target interface 440, and concentrates the interference wave 42.

The diaphragm 462 of the target interface 440 communicates with the internal chamber 452 in the catheter 450 of the target interface 440, and is contactable at least in close proximity to the neoplastic target 44 when the neoplastic target 44 is contained in a lumen in a body of a patient.

The target interface 440 further includes a liquid 464. The liquid 464 of the target interface 440 is contained in the internal chamber 452 in the catheter 450 of the target interface 440, communicates with the first transducer 28, the second transducer 32, the n^{th} transducer 36, and the diaphragm 462 of the target interface 440, and functions as an acoustical coupler to combine the first waveform 30, the second waveform 34, and the n^{th} waveform 38 to form the interference wave 42.

The target interface 440 further includes a steering tube 466. The steering tube 466 of the target interface 440 enters the internal chamber 452 in the catheter 450 of the target interface 440 through a port 468 in the catheter 450 of the target interface 440, and extends to the distal treatment end 456 of the catheter 450 of the target interface 440, with a proximal end 470 of the steering tube 466 of the target interface 440 disposed externally to the internal chamber 452 in the catheter 450 of the target interface 440.

The target interface 440 further includes a steering apparatus 472. The steering apparatus 472 of the target interface 440 passes through the steering tube 466 of the target interface 440, and functions to steer the catheter 450 of the target interface 440 through the lumen in the body of the patient to at least close proximity to the neoplastic target 44.

The steering apparatus 472 of the target interface 440 is operatively connected to the controller 18, and can preferably be bimetallic, but is not limited to that.

The target interface 440 further includes a viewing tube 474. The viewing tube 474 of the target interface 440 enters the internal chamber 452 in the catheter 450 of the target interface 440 through a port 476 in the catheter 450 of the target interface 440, and extends to the distal treatment end 456 of the catheter 450 of the target interface 440, with a proximal end 478 of the viewing tube 474 of the target interface 440 disposed externally to the internal chamber 452 in the catheter 450 of the target interface 440.

The target interface 440 further includes a viewing apparatus 480. The viewing apparatus 480 of the target interface 440 passes through the viewing tube 474 of the target interface 440, and functions to assist steering the steering apparatus 472 of the target interface 440 and viewing the neoplastic target 44 being damaged.

The viewing apparatus 480 of the target interface 440 can preferably be fiber optical, but is not limited to that.

### H. The Impressions.

It will be understood that each of the elements described above or two or more together may also find a useful application in other types of constructions differing from the types described above.

While the embodiments of the present invention have been illustrated and described as embodied in a neoplasm cell destruction device utilizing low frequency sound waves to disrupt or displace cellular materials, however, they are not limited to the details shown, since it will be understood that various omissions, modifications, substitutions, and changes in the forms and details of the device illustrated and its operation can be made by those skilled in the art without departing in any way from the spirit of the embodiments of the present invention.

Without further analysis, the foregoing will so fully reveal the gist of the embodiments of the present invention that others can by applying current knowledge readily adapt them for various applications without omitting features that from the standpoint of prior art fairly constitute characteristics of the generic or specific aspects of the embodiments of the present invention.

The invention claimed is:

1. A neoplasm cell destruction device utilizing low frequency sound waves to do at least one of disrupt and displace cellular materials in neoplastic cells having resonant frequencies, said device comprising:
a) a plurality of signal generators;
b) a controller;
c) a plurality of amplifiers;
d) a plurality of transducers; and
e) a target interface;
wherein each signal generator generates a signal;
wherein said controller is in electrical communication with said plurality of signal generators;
wherein said controller generates timing and control signals for selectively activating said plurality of signal generators;
wherein each amplifier is in electrical communication with a respective signal generator;
wherein each amplifier amplifies said signal generated by said respective signal generator so as to form an amplified signal;
wherein each transducer is in electrical communication with a respective amplifier;
wherein each transducer is driven by said amplified signal formed by said respective amplifier;
wherein each transducer forms a waveform that is a low frequency sound wave;
wherein said target interface combines said waveforms formed by said plurality of transducers to form an interference wave;
wherein said interference wave is a low frequency sound wave;
wherein said interference wave is impactable upon the neoplastic cells and damages and ultimately destructs the neoplastic cells;
wherein said target interface includes a hollow and hand-held body;
wherein said hollow and hand-held body of said target interface contains an internal chamber; and
wherein said hollow and hand-held body of said target interface is holdable in a hand of a user and is contactable at least in close proximity to the neoplastic target when the neoplastic target is contained in a large area in a body of a patient.

2. The device of claim 1, wherein said controller is a microprocessor.

3. The device of claim 1, further comprising a user interface; and
wherein said user interface is in electrical communication with said controller.

4. The device of claim 3, wherein said user interface is at least one of a keyboard and a display.

5. The device of claim 1, further comprising a feedback sensor;
wherein said feedback sensor is disposed in close proximity to said target interface;
wherein said feedback sensor is in electrical communication with said controller;
wherein said feedback sensor receives a feedback wave emanating from the neoplastic cells when the neoplastic cells are impacted upon by said interference wave and generates a feedback signal in response thereto; and
wherein said feedback signal is received by said controller that in turn continually compares the feedback signal received to said interference wave and automatically adjusts each signal generator until said interference wave is at the resonant frequencies of the neoplastic cells so as to maximize damage to the neoplastic cells.

6. The device of claim 1, further comprising a feedback sensor;
wherein said feedback sensor is disposed in close proximity to said target interface;
wherein said feedback sensor is in electrical communication with each said signal generator;
wherein said feedback sensor receives a feedback wave emanating from the neoplastic cells when the neoplastic cells are impacted upon by said interference wave and generates a feedback signal in response thereto; and
wherein said feedback signal is received by each said signal generator that in turn are manually adjusted until said interference wave is at the resonant frequencies of the neoplastic cells so as to maximize damage to the neoplastic cells.

7. The device of claim 1, wherein said hollow and hand-held body of said target interface has an upper wall;
wherein said hollow and hand-held body of said target interface has a lower treatment wall;
wherein said hollow and hand-held body of said target interface has a loop;
wherein said loop of said target interface is accessible from said upper wall of said hollow and hand-held body of said target interface; and
wherein said loop of said target interface has the hand of the user passable therethrough.

8. The device of claim 1, wherein said hollow and hand-held body of said target interface is at least one of plastic and metal.

9. The device of claim 7, wherein said first transducer, said second transducer, and said n^{th} transducer are disposed at said hollow and hand-held body of said target interface, with said first waveform, said second waveform, and said n^{th} waveform emanating therefrom into said internal chamber in said hollow and hand-held body of said target interface.

10. The device of claim 7, wherein said target interface includes a diaphragm;
wherein said diaphragm of said target interface is disposed at said lower treatment wall of said hollow and hand-held body of said target interface;
wherein said diaphragm of said target interface closes said lower treatment wall of said hollow and hand-held body of said target interface; and
wherein said diaphragm of said target interface distributes said interference wave.

11. The device of claim 1, wherein said diaphragm of said target interface communicates with said internal chamber in said hollow and hand-held body of said target interface; and
wherein said diaphragm of said target interface is contactable at least in close proximity to the neoplastic target when said neoplastic target is contained in the large area in the body of the patient.

12. The device of claim 10, wherein said target interface includes a liquid;
wherein said liquid of said target interface is contained in said internal chamber in said hollow and hand-held body of said target interface;
wherein said liquid of said target interface communicates with said first transducer, said second transducer, said n^{th} transducer, and said diaphragm of said target interface; and
wherein said liquid of said target interface functions as an acoustical coupler to combine said first waveform, said second waveform, and said n^{th} waveform to form said interference wave.
